(19) United States
(12) Patent Application Publication
(10) Pub. No.: US 2008/0038386 A1
(43) Pub. Date: Feb. 14, 2008

(54) Use of a liquid cooling sensate to provide a pungent, warming or tingling sensate composition

(76) Inventors: Ashok K Moza, Baytown, TX (US); Stephen E. Rohde, Baytown, TX (US); John Charles Leffingwell, Baytown, TX (US); Diane Leffingwell, Baytown, TX (US)

Correspondence Address: Qaroma Inc.;Subsidiary of Chemicals Incorporated, 12321 Hatcherville Road, Baytown, TX, 77521, US

(21) Appl. No.: 11/497,479

(22) Filed: Aug. 2, 2006

(51) Int. Cl. A61K 36/81; A61K 36/31; A61K 31/21; A61K 31/26; A61K 31/165

(52) U.S. Cl. 424/755; 514/514; 424/769; 424/760; 424/773; 514/617

(57) ABSTRACT

The present invention is directed to a sensate composition consisting of a liquid cooling composition, and at least one warming, tingling, or pungent sensate ingredient of either synthetic or natural origin which provides enhanced warming, tingling, or pungent properties. The liquid cooling sensate of the invention is a eutectic mixture of 2-Isopropyl-N,2,3-trimethylbutyramide and N-Ethyl-p-menthane-3-carboxamide, or at least one cooling agent selected from the group consisting of N-(2-hydroxyethyl)-2,3-dimethyl-2-isopropylbutanamide, N-(3-ethoxypropyl)-2,3-dimethyl-2-isopropylbutanamide, N-(3-isopropoxypropyl)-2,3-dimethyl-2-isopropylbutanamide, N-(3-butoxypropyl)-2,3-dimethyl-2-isopropylbutanamide,N-Ethyl-2,2-diisopropylbutanamide, N-(1,1-dimethyl-2-hydroxyethyl)-2,2-diethylbutanamide, N-(2-ethoxyethyl)-2,3-dimethyl-2-isopropylbutanamide and N-(3-methoxypropyl)-2,3-dimethyl-2-isopropylbutanamide. The warming, tingling, or pungent sensate of the invention consists of at least one component that is an isothiocyanate and/or an amide or a natural product that contains at least one warming, tingling, or pungent principle that is an isothiocyanate and/or an amide. The present invention is further directed to a method of using the sensate composition in a food, pharmaceutical or personal care product.

### BACKGROUND OF THE INVENTION

The present invention relates to a composition imparting an increased sensation of warming, tingling or pungency. More specifically, the present invention is a composition including a liquid cooling sensate that is a eutectic mixture of 2-Isopropyl-N,2,3-trimethylbutyramide and N-Ethyl-p-menthane-3-carboxamide and a warming, tingling, or pungent sensate preferably comprised of at least one component that is, or contains, an isothiocyanate and/or an amide or other known warming, tingling, or pungent materials. Such warming, tingling, or pungent sensates may be individual chemical compounds or mixtures of synthetic and/or of natural origin.

There is considerable interest in the flavor and fragrance industry in the use of compositions that provide warming, tingling or pungency as such materials provide interesting sensorial experiences in consumer products such as personal care products (perfumes deodorants, cosmetics, shampoos, skin creams, toothpastes and the like), pharmaceuticals (such as cough syrups, cough drops and the like) as well as foods and beverages (such as chewing gum, soda and the like).

Materials and/or applications or potential applications that provide such interesting sensorial sensations are described in laid open United States Patent Application document numbers 20030152682, 20030072842, 20040141927, 20040202619, 20040202760, 20040241312, 20050010062, 20050074533, 20050075368, 20050187211, 20050202118, 20050197387, 20050208084, 20060024425, 20060034936, 20060057268, 20060068071, 20060062872, 20060051456, 20060045934, 20060045851, 20060034936, 20060034894, 20060024245, 20060024244, 2005233042, 20050260266, 20050203473, 20050192348, 20050169852, 20050163727, 20050137109, 20050106111, 20050084551, 20050058602, 20050025721, 20050002876, 20040258633, 20040247647, 20040247646; 20040220087, 20040101494, 20040101493, 20040101492, 20040082928, 20030211051, 20030206874, 20030168375, 20030158111, 20030152525, 20030139437, 20030129143, 20030124066. 20030124065, 20030118613, 20030104020 20030103914, 20030091600, 20030049303, 20030039617, 20030012743, 20030008062, 20030007997, 20030007937, 20030003219, 20030003140, 20020197215, 20020173436, 20020142015, 20020119231, 20020086878, 20020082307, 20020076421, 20020009478, 20010016577, 20010006624 and U.S. Pat. Nos. 6,899,901, 6,890,567, 6,846,495, 6,838,106, 6,780,443, 6,746,697, 6,740,311, 6,365,215, 6,159,509, 5,753,609, 5,545,424, 5,407,665. 6,726,897, 6,719,962, 6,706,277, 6,706,256, 6,703,000, 6,702,999, 6,682,722, 6,673,844, 6,350,438, 6,280,762, 6,264,924, 6,117,418 and PCT WO 2005/004635 (A1), PCT WO 2006/003210 (A1), and PCT WO 2006/009425 (A1).

It is known that by combining certain compounds that possess flavor and/or sensate properties can produce new active ingredients systems having altered sensory properties. For example, in PCT published application WO 98/47482, formulations are disclosed for cough drops which include a physiological cooling agent (such as menthol, n-N-substituted-p-menthane-3-carboxamides, acyclic tertiary and secondary carboxamides, 3-1-menthoxy propan-1,2-diol) and a physiological warming agent (such as vanillyl alcohol n-butyl ether, vanillyl alcohol n-propyl ether, vanillyl alcohol isopropyl ether, vanillyl alcohol isobutyl ether, vanillyl alcolol n-amino ether, vanillyl alcohol isoamyl ether, vanillyl alcohol n-hexyl ether vanillyl alcohol methyl ether, vanillyl alcohol ethyl ether, gingerol, shogaol, paradol, zingerone, capsaicin, dihydrocapsaicin, nordihydrocapsaicin, homocapsaicin, homodihydrocapsaicin, ethanol, benzyl alcohol, chloroform, eugenol, cinnamon oil, cinnamic aldehyde) can provide compositions possessing modified sensate properties.

It further is known that certain materials (e.g., alkadieneamides & alkatrieneamides), which at higher levels of use may possess pungent, burning or tingling sensate properties, may exhibit different flavor effects when used at lower application levels. Such an important alkatrienamide is Spilanthol, the pungency of which is disclosed in Nakatani et al “Pungent Alkamides from Spilanthes acmella L. var. oleracea Clarke, Biosci. Biotech. Biochem., 56(5), 759-762 (1992). For example, in laid open United States Patent Application document 20040241312, it is stated that” Among a long chain fatty acid alkyl amides, 2E,6Z,8E-decatrienoic acid-N-isobutylamide (spilanthol) has been described as strongly salivation inducing and tingly or prickly. Spilanthol is however at the same time above all spicy-hot and strongly numbing as well as an astringent (Lebensm.-Wiss. U. Technol. 1992, 25, 417-421).” But at 30 ppm in sucrose solution, spilanthol is described as “tingling & mouth watering” and is not reported to impart “burning, pungency, scratchy, numbing, warming” effects (Ley, et. al., 11th Weurmann Flavour Research Symposium, 2005, Roskilde, Denmark). Other “tingling” amides such as (2E,6Z)-N-cyclopropylnona-2,6-dienamide and (2E,6Z)-N-ethyldodeca-2,6-dienamide also possess “umami” taste characteristics (United States Patent Application document 20040202619).

### OBJECTS AND SUMMARY OF THE INVENTION

1. It is an object of the present invention to provide a sensate composition that provides a tingling and/or stinging warming pungent impression upon contact.
2. It is a further object of the present invention to provide a sensate composition consisting of a eutectic liquid mixture of 2-Isopropyl-N,2,3-trimethylbutyramide (WS-23) and N-Ethyl-p-menthane-3-carboxamide (WS-3), or at least one cooling agent selected from the group consisting of N-(2-hydroxyethyl)-2,3-dimethyl-2-isopropylbutanamide, N-(3-ethoxypropyl)-2,3-dimethyl-2-isopropylbutanamide, N-(3-isopropoxypropyl)-2,3-dimethyl-2-isopropylbutanamide, N-(3-butoxypropyl)-2,3-dimethyl-2-isopropylbutanamide, N-Ethyl-2,2-diisopropylbutanamide, N-(1,1-dimethyl-2-hydroxyethyl)-2,2-diethylbutanamide, N-(2-ethoxyethyl)-2,3-dimethyl-2-isopropylbutanamide and N-(3-methoxypropyl)-2,3-dimethyl-2-isopropylbutanamide, which in combination with an isothiocyanate, or a natural material containing such isothiocyanates, provides an enhanced tingling and/or stinging, warming pungent impression upon contact.
3. It is a further object of the present invention to provide a sensate composition consisting of a eutectic liquid mixture of 2-Isopropyl-N,2,3-trimethylbutyramide (WS-23) and N-Ethyl-p-menthane-3-carboxamide (WS-3), or at least one cooling agent selected from the group consisting of N-(2-hydroxyethyl)-2,3-dimethyl-2-isopropylbutanamide, N-(3-ethoxypropyl)-2,3-dimethyl-2-isopropylbutanamide, N-(3-isopropoxypropyl)-2,3-dimethyl-2-isopropylbutanamide, N-(3-butoxypropyl)-2,3-dimethyl-2-isopropylbutanamide,N-Ethyl-2,2-diisopropylbutanamide, N-(1,1-dimethyl-2-hydroxyethyl)-2,2-diethylbutanamide, N-(2-ethoxyethyl)-2,3-dimethyl-2-isopropylbutanamide and N-(3-methoxypropyl)-2,3-dimethyl-2-isopropylbutanamide, which in combination with an amide possessing tingling and/or stinging, warming pungent sensory properties, or a natural product containing such amides, provides an enhanced tingling and/or stinging, warming pungent impression upon contact.

The use of various cooling agents, including 2-Isopropyl-N,2,3-trimethylbutyramide (WS-23) or N-Ethyl-p-menthane-3-carboxamide (WS-3), in combination with certain amides and vanillyl ethers having tingling and/or stinging or warming or pungent sensory properties (such as Spilanthol, capsaicin or vanillyl butyl ether), or natural materials containing such agents, to provide sensate compositions is known.

However, we have now surprisingly found that utilization of the liquid eutectic mixture of 2-Isopropyl-N,2,3-trimethylbutyramide (WS-23) or N-Ethyl-p-menthane-3-carboxamide (WS-3), sold under the tradename of ICE 1500, or at least one cooling agent selected from the group consisting of N-(2-hydroxyethyl)-2,3-dimethyl-2-isopropylbutanamide (sold under the tradename of ICE 6000), N-(3-ethoxypropyl)-2,3-dimethyl-2-isopropylbutanamide (sold under the tradename of ICE 7000), N-(3-isopropoxypropyl)-2,3-dimethyl-2-isopropylbutanamide (sold under the tradename of ICE 8000), N-(3-butoxypropyl)-2,3-dimethyl-2-isopropylbutanamide (sold under the tradename of ICE 9000), N-Ethyl-2,2-diisopropylbutanamide (ICE 10000), N-(1,1-dimethyl-2-hydroxyethyl)-2,2-diethylbutanamide (sold under the tradename of ICE 11000), N-(2-ethoxyethyl)-2,3-dimethyl-2-isopropylbutanamide (sold under the tradename of ICE 12000) and N-(3-methoxypropyl)-2,3-dimethyl-2-isopropylbutanamide (sold under the tradename of ICE 13000), provides improved sensory properties while providing enhanced pungency to products containing pungent amides such as capsaicin. Such pungency enhancement is about 25-35% in the case of capsaicin containing products. While N-Ethyl-p-menthane-3-carboxamide (WS-3) alone also provides similar pungency enhancement, an unbalanced scratchy-irritating and distorted sensory impression accompanies this enhancement. Utilization of the aforementioned eutectic cooling mixture has now been found to provide the desired pungency enhancement with no sensory imbalance or distortion.

We have further surprisingly found that by utilizing a liquid eutectic mixture of 2-Isopropyl-N,2,3-trimethylbutyramide (WS-23) or N-Ethyl-p-menthane-3-carboxamide (WS-3), or at least one cooling agent selected from the group consisting of N-(2-hydroxyethyl)-2,3-dimethyl-2-isopropylbutanamide, N-(3-ethoxypropyl)-2,3-dimethyl-2-isopropylbutanamide, N-(3-isopropoxypropyl)-2,3-dimethyl-2-isopropylbutanamide, N-(3-butoxypropyl)-2,3-dimethyl-2-isopropylbutanamide, N-Ethyl-2,2-diisopropylbutanamide, N-(1,1-dimethyl-2-hydroxyethyl)-2,2-diethylbutanamide, N-(2-ethoxyethyl)-2,3-dimethyl-2-isopropylbutanamide and N-(3-methoxypropyl)-2,3-dimethyl-2-isopropylbutanamide, in conjunction with isothiocyanates, such as allyl isothiocyanate, or a natural material containing such an isothiocyanate, that a dramatic increase in pungency is observed. Such pungency enhancement is about 90-110% in the case of allyl isothiocyanate with no sensory imbalance. Such a degree of pungency enhancement for isothiocyanate containing materials is a new finding.

In an embodiment of the present invention, a method of using such sensate compositions as a fragrance or a flavor is provided, which includes forming a sensate composition having at least one cooling sensate, one of which consists of a eutectic mixture of 2-Isopropyl-N,2,3-trimethylbutyramide (WS-23) or N-Ethyl-p-menthane-3-carboxamide (WS-3), or at least one cooling agent selected from the group consisting of N-(2-hydroxyethyl)-2,3-dimethyl-2-isopropylbutanamide, N-(3-ethoxypropyl)-2,3-dimethyl-2-isopropylbutanamide, N-(3-isopropoxypropyl)-2,3-dimethyl-2-isopropylbutanamide, N-(3-butoxypropyl)-2,3-dimethyl-2-isopropylbutanamide,N-Ethyl-2,2-diisopropylbutanamide, N-(1,1-dimethyl-2-hydroxyethyl)-2,2-diethylbutanamide, N-(2-ethoxyethyl)-2,3-dimethyl-2-isopropylbutanamide and N-(3-methoxypropyl)-2,3-dimethyl-2-isopropylbutanamide, and at least one warming, tingling, or pungent sensate material. Such sensate compositions can be used in finished consumer products or be concentrated compositions that are used as flavor and/or fragrance ingredients.

Additionally, as will be obvious to those skilled in the art, such sensate compositions can be designed for use in non-lethal personal defense devices or non-lethal warfare devices, as alternatives to purely capsicum based materials, for temporarily incapacitating assailants or combatants.

The above, and other objects, features and advantages of the present invention will become apparent from the following description. However, these examples are not to be construed to limit the scope of the invention.

### DETAILED DESCRIPTION OF THE PREFERRED EMBODIMENT

As described herein, 2-Isopropyl-N,2,3-trimethylbutyramide (WS-23) or N-Ethyl-p-menthane-3-carboxamide (WS-3), or at least one cooling agent selected from the group consisting of N-(2-hydroxyethyl)-2,3-dimethyl-2-isopropylbutanamide, N-(3-ethoxypropyl)-2,3-dimethyl-2-isopropylbutanamide, N-(3-isopropoxypropyl)-2,3-dimethyl-2-isopropylbutanamide, N-(3-butoxypropyl)-2,3-dimethyl-2-isopropylbutanamide, N-Ethyl-2,2-diisopropylbutanamide, N-(1,1-dimethyl-2-hydroxyethyl)-2,2-diethylbutanamide, N-(2-ethoxyethyl)-2,3-dimethyl-2-isopropylbutanamide and N-(3-methoxypropyl)-2,3-dimethyl-2-isopropylbutanamide, and other compounds which are known cooling agents. In addition, amides (such as capsaicin), isothiocyanates (such as allyl isothiocyanates) and other compounds described herein are warming, tingling, or pungent sensate materials.

Preferably, the liquid cooling sensate of this invention is a stable eutectic liquid mixture of 2-Isopropyl-N,2,3-trimethylbutyramide (WS-23) and N-Ethyl-p-menthane-3-carboxamide (WS-3) with a weight ratio of 2-Isopropyl-N,2,3-trimethylbutyramide to N-Ethyl-p-menthane-3-carboxamide in the range of 60:40 to 30:70 with the corresponding crystallization point being below 15° C. Most preferred is the liquid mixture of 2-Isopropyl-N,2,3-trimethylbutyramide (WS-23) and N-Ethyl-p-menthane-3-carboxamide (WS-3) with a weight ratio of 2-Isopropyl-N,2,3-trimethylbutyramide to N-Ethyl-p-menthane-3-carboxamide of 1:1 with a crystallization point being below 0° C.

Other preferred cooling agents are at least one cooling agent selected from the group consisting of N-(2-hydroxyethyl)-2,3-dimethyl-2-isopropylbutanamide, N-(3-ethoxypropyl)-2,3-dimethyl-2-isopropylbutanamide, N-(3-isopropoxypropyl)-2,3-dimethyl-2-isopropylbutanamide, N-(3-butoxypropyl)-2,3-dimethyl-2-isopropylbutanamide,N-Ethyl-2,2-diisopropylbutanamide, N-(1,1-dimethyl-2-hydroxyethyl)-2,2-diethylbutanamide, N-(2-ethoxyethyl)-2,3-dimethyl-2-isopropylbutanamide and N-(3-methoxypropyl)-2,3-dimethyl-2-isopropylbutanamide.

Other known cooling agents that may be optionally employed are (−)-(3S,3aS,3bR,4S,7R,7aR)-4-isopropyl-3,7-dimethyloctahydro-1H-cyclopenta[1,3]cyclopropa[1,2]benzen-3-ol (Cubebol), isopulegol, 3-(1-menthoxy)propan-1,2-diol, p-menthan-3,8-diol, 6-isopropyl-9-methyl-1,4-dioxaspiro-(4,5)-decane-2-methanol (Frescolat MGA), menthyl lactate (Frescolat ML), menthyl succinate, alkaline earth salts of menthyl succinate, 3,5,5-trimethylcyclohexanol, ethyl 2-((1R,2S,5R)-2-isopropyl-5-methylcyclohexanecarboxamido)acetate (WS-5), menthol propylene glycol carbonate (Frescolat MPC), monomenthyl glutarate, 2-{[(1R,2S,5R)-2-isopropyl-5-methylcyclohexyl]oxy}ethanol, menthyl pyrrolidone carboxylate (Questice), N,N-Dimethyl menthyl succinamide, Menthyl 3-hydroxybutyrate, Menthol ethylene glycol carbonate (Frescolat MGC), (1R,3R,4S)-3-menthyl 3,6-dioxaheptanoate, (1R,2S,5R)-3-menthyl methoxyacetate, (1R,2S,5R)-3-menthyl 3,6,9-trioxadecanoate, (1R,2S,5R)-3-menthyl 3,6,9-trioxadecanoate, (1R,2S,5R)-3-menthyl (2-hydroxyethoxy)acetate, (1R,2S,5R)-menthyl 11-hydroxy-3,6,9-trioxaundecanoate, 1-[2-hydroxyphenyl]-4-[2-nitrophenyl-]-1,2,3,6-tetrahydropyrimidine-2-one (Icilin, also known as AG-3-5), 3-[(2-isopropyl-5-methylcyclohexyl)oxy]-2-methylpropane-1,2-diol, N-(2-ethoxyethyl)-2,3-dimethyl-2-isopropylbutyramide, N-(3-methoxypropyl)-2,3-dimethyl-2-isopropylbutyramide, N-(3-ethoxypropyl)-2,3-dimethyl-2-isopropylbutyramide, N-(3-isopropoxypropyl)-2,3-dimethyl-2-isopropylbutyramide, N-(3-butoxypropyl)-2,3-dimethyl-2-isopropylbutyramide, 2-(5′-methyl-2′-(methylethyl)cyclohexyloxy)ethan-1-ol, 3-(5′-methyl-2′-(methylethyl)cyclohexyloxy)propan-1-ol, 4-(5′-methyl-2′-(methylethyl)cyclohexyloxy)butan-1-ol, menthol, peppermint oil, commint oil, menthone & eucalyptol.

Preferably, at least one of the warming, tingling, or pungent sensate materials is an appropriate isothiocyanate compound such as allyl isothiocyanate, phenylethyl isothiocyanate, Benzyl isothiocyanate, Butyl isothiocyanate, 3-Methylthiopropyl isothiocyanate, 2-Phenylethyl isothiocyanate, Pentyl isothiocyanate, 3-Butenyl isothiocyanate, sec-Butyl isothiocyanate, 5-Hexenyl isothiocyanate, Hexyl isothiocyanate, Isoamyl isothiocyanate, Isobutyl isothiocyanate, Isopropyl isothiocyanate, Lesquerellin (6-methylthiohexyl isothiocyanate), 4-pentenyl isothiocyanate, Erucin (4-methylthiobutyl isothiocyanate), Berteroin (5-methylthiopentyl isothiocyanate), Ethyl isothiocyanate, Methyl isothiocyanate or natural products containing such isothiocyanates (or their Glucosinolate precursors), including Brassica oleracea var. capitata (common cabbage), Brassica rapa var. Pekinensis (Pe-tsai Chinese cabbage), Brassica rapa var. chinensis (Pak-choi, Chinese cabbage), Nasturtium officinale (Watercress), Brassica juncea (Brown or Oriental mustard), Brassica hirta (Yellow mustard), Wasabi japonica (Japanese Horseradish), Brassica rapa septiceps (Turnip), Brassica oleracea botrytis (Cauliflower), Cochlearia armoracia or Armoracia rusticana (Horseradish), Brassica oleracea gemmifera (Brussels sprout), Raphanus sativus (Radish), Raphanus raphanistrum (Wild radish) or other isothiocyanate containing botanicals. When such botanicals are used the may be in the form of the raw or dried botanical, oils distilled from the botanical, extractives of the botanical, or processed foods containing the botanical.

Other known pungent, warming or tingling agents that may be employed, alone or in combination, are vanillyl methyl ether, vanillyl ethyl ether, vanillyl propyl ether, vanillyl isopropyl ether, vanillyl butyl ether, vanillin propylene glycol acetal, ethyl vanillin propylene glycol acetal, 4-(1-menthoxymethyl)-2-phenyl-1,3-dioxolane, 4-(1-menthoxymethyl)-2-(3′,4′-dihydroxy-phenyl)-1,3-dioxolane, 4-(1-menthoxymethyl)-2-(2′-hydroxy-3′-methoxy-phenyl)-1,3-dioxolane, 4-(1-menthoxymethyl)-2-(4′-methoxyphenyl)-1,3-dioxolane, 4-(1-menthoxymethyl)-2-(3′4′-methylenedioxy-phenyl)-1,3-dioxolane, 4-(1-menthoxymethyl)-2-(3′-methoxy-4′-hydroxyphenyl)-1,3-dioxolane, 1′-acetoxychavicol acetate, 1′-acetoxyeugenol acetate, 4-(butoxymethyl)-2-methoxyphenyl acetate, 4-(hexyloxymethyl)-2-methoxyphenyl acetate, cinnamaldehyde, eugenol, nicotinaldehyde, methyl nicotinate, propyl nicotinate, 2-butoxyethyl nicotinate, benzyl nicotinate, pungent sesquiterpenoid dialdehydes (such as polygodial, isodrimeninol, warburganal, isovelleral, velleral, and merulidial), (2S,3R,4R)-3,4-bis(1,3-benzodioxol-5-ylmethyl)tetrahydrofuran-2-ol (cubebin), (2E,6Z,8E)-N-isobutyideca-2,6,8-trienamide (affinin or spilanthol), (2E,4E,8Z)-N-isobutyldeca-2,6,8-trienamide (isoaffinin), (2E,6Z,8E)-N-(2-methylbutyl)deca-2,6,8-trienamide (homospilanthol ), (2E,6Z)-N-cyclopropylnona-2,6-dienamide, (2E,6Z)-N-ethylnona-2,6-dienamide, N-isobutyl-(2E,4E)-decadienamide (trans-Pellitorine), (2E,4Z)-N-isobutybdeca-2,4-dienamide (cis-Pellitorine), (2E)-N-isobutyidec-2-en-4-ynamide (dehydropellitorine), (2E,4E)-N-(2-methylbutyl)deca-2,4-dienamide (homopellitorine), (2E,4E)-N-[(2S)-2-methylbutylideca-2,4-dienamide [(2′S)-homopellitorine], (2E,4Z)-N-[(2S)-2-methylbutyl]deca-2,4-dienamide [(2′S)-cis-homopellitorine], (2E,7Z,9E)-N-isobutylundeca-2,7,9- trienamide, (2E,4E,8Z)-N-isobutylundeca-2,4,8- trienamide, (2E,4E,8Z)-N,N-dimethylundeca-2,4,8- trienamide, (2E,8Z)-N-cyclopropylundeca-2,8-dienamide, (2E,8Z)-N-ethylundeca-2,8-dienamide, (2E,4E,8Z)-N-(2-hydroxy-2-methylpropyl)deca-2,4,8-trienamide, (2E ,4Z,8Z)-N-(2-hydroxy-2-methylpropyl)deca-2,4,8-trienamide, (2E)-N-isobutyldec-2-enamide, (2E)-N-(2-methylbutyl)dec-2-enamide, N-isobutyldecanamide, N-heptyl-2-hydroxy-2-(4-hydroxyphenyl)acetamide, N-octyl-hydroxyphenyl)acetamide, 2-hydroxy-2-(4-hydroxyphenyl)acetamide, N-nonyl-2-hydroxy-2-(4-N-heptyl-2-hydroxy-2-(4-methoxyphenyl)acetamide, N-octyl-2-hydroxy-2-(4-methoxyphenyl)acetamide, N-nonyl-2-hydroxy-2-(4-methoxyphenyl)acetamide, N-heptyl-2-hydroxy-2-(3-hydroxy-4-methoxyphenyl)acetamide, N-nonyl-2-hydroxy-2-(3-hydroxy-4-methoxyphenyl)acetamide, N-octyl-2-hydroxy-2-(3,4-dihydroxyphenyl)acetamide, N-heptyl-2-hydroxy-2-(4-hydroxy-3-methoxyphenyl)acetamide, N-nonyl-2-hydroxy-2-(4-hydroxy-3-methoxyphenyl)acetamide, N-(7-methyl-1-octyl)-2-hydroxy-2-(4-hydroxy-3-methoxyphenyl)acetamide, (6E)-N-(4-hydroxy-3-methoxybenzyl)-8-methylnon-6-enamide (capsaicin), N-(4-hydroxy-3-methoxybenzyl)-8-methyinonanamide (dihydrocapsaicin), (E)-N-(4-hydroxy-3-methoxybenzyl)-9-methyldec-7-enamide (homocapsaicin), (E)-N-(4-hydroxy-3-methoxybenzyl)-8-methyldec-6-enamide (homocapsaicin II), N-(4-hydroxy-3-methoxybenzyl)-9-methyldecanamide (dihydrohomocapsaicin), N-(4-hydroxy-3-methoxybenzyl)-8-methyidecanamide (dihydrohomocapsaicin II), N-(4-hydroxy-3-methoxybenzyl)-7-methyloctanamide (dihydronorcapsaicin), N-(4-hydroxy-3-methoxybenzyl)nonanamide, N-(4-hydroxy-3-methoxybenzyl)octanamide, (2E,6Z,8E, 10E)-N-isobutyldodeca-2,6,8,10-tetraenamide (alpha-sanshool), (2E ,6Z,8E,10E)-N-(2-hydroxy-2-methylpropyl)dodeca-2,6,8,10-tetraenamide (hydroxy-alpha-sanshool or sanshoamide), (2E,6E,8E,10E)-N-isobutyldodeca-2,6,8,10-tetraenamide (beta-sanshool), (2E,6E,8E,10E)-N-(2-hydroxy-2-methylpropyl)dodeca-2,6,8,10-tetraenamide (hydroxy-beta-sanshool), (2E,4E,8E,10E,12E)-N-isobutyltetradeca-2,4,8,10,12-pentaenamide (delta-sanshool), (2E,4E,8Z,10E,12E)-N-isobutyltetradeca-2,4,8,10,12-pentaenamide (gamma-sanshool), 5-hydroxy-1-(4-hydroxy-3-methoxyphenyl)decan-3-one ([6]-gingerol), 5-hydroxy-1-(4-hydroxy-3-methoxyphenyl)dodecan-3-one ([8]-gingerol), 5-hydroxy-1-(4-hydroxy-3-methoxyphenyl)tetradecan-3-one ([10]-gingerol), (4E)-1-(4-hydroxy-3-methoxyphenyl)dec-4-en-3-one ([6]-shogaol), (4E)-1-(4-hydroxy-3-methoxyphenyl)dodec-4-en-3-one ([8]-shogaol), (4E)-1-(4-hydroxy-3-methoxyphenyl)tetradec-4-en-3-one ([10]-shogaol), 1-(4-hydroxy-3-methoxyphenyl)nonan-3-one ([5]-paradol), 1-(4-hydroxy-3-methoxyphenyl)decan-3-one ([6]-paradol), 1-(4-hydroxy-3-methoxyphenyl) undecan-3-one ([7]-paradol), 1-(4-hydroxy-3-methoxyphenyl)dodecan-3-one ([8]-paradol), 1-(4-hydroxy-3-methoxyphenyl)tetradecan-3-one ([10]-paradol), 4-(4-hydroxy-3-methoxyphenyl)butan-2-one (zingerone), (E)-N-(4-hydroxy-3-methoxyphenethyl)-3-(4-hydroxy-3-methoxyphenyl)acrylamide (trans-feruloylmethoxytyramine), (Z)-N-(4-hydroxy-3-methoxyphenethyl)-3-(4-hydroxy-3-methoxyphenyl)acrylamide (cis-feruloylmethoxytyramine), N-(4-hydroxy-3-methoxyphenethyl)-3-(4-hydroxy-3-methoxyphenyl)propanamide (dihydroferuloylmethoxytyramine), (E)-N-(3,4-dihydroxyphenethyl)-3-(4-hydroxy-3-methoxyphenyl)acrylamide (trans-feruloyldopamine), (Z)-N-(3,4-dihydroxyphenethyl)-3-(4-hydroxy-3-methoxyphenyl)acrylamide (cis-feruloyldopamine), (E)-N-(4-hydroxyphenethyl)-3-(3,4-dihydroxyphenyl)acrylamide (trans-caffeoyltyramine), (Z)-N-(4-hydroxyphenethyl)-3-(3,4-dihydroxyphenyl)acrylamide (cis-caffeoyltyramine), (E)-N-(3,4-dimethoxyphenethyl)-3-(3,4-dimethoxyphenyl)acrylamide (trans-rubenamine), (Z)-N-(3,4-dimethoxyphenethyl)-3-(3,4-dimethoxyphenyl)acrylamide (cis-rubenamine), (E)-N-(3,4-dimethoxyphenethyl)-3-(3,4-dimethoxyphenyl)-N-methylacrylamide (rubenamide), (E)-3-(1,3-benzodioxol-5-yl)-N-[2-(1,3-benzodioxol-5-yl)ethyl]-N-methylacrylamide (dioxamide), (E)-3-(1,3-benzodioxol-5-yl)-N-[2-(1,3-benzodioxol-5-yl)ethyl]acrylamide (dioxamine), (E)-N-[2-(1,3-benzodioxol-5-yl)ethyl]-N-methyl-3-phenylacrylamide (zanthomamide), 1-[(2E,4E)-deca-2,4-dienoyl]piperidine (achilleamide), 1-[(2E)-dec-2-enoyl]piperidine (dihydroachilleamide), 1-[(2E,4E)-5-(1,3-benzodioxol-5-yl)penta-2,4-dienoyl]piperidine (piperine), 1-[(2E,4E)-deca-2,4-dienoyl]pyrrolidine (sarmentine), (2E,4E)-N-butyldeca-2,4-dienamide, (2E,4E)-N-(3-methylbutyl)deca-2,4-dienamide, (2E,4E)-N-(2-hydroxyethyl)deca-2,4-dienamide, (2E,4E)-N-(2-ethylhexyl)deca-2,4-dienamide or natural products containing these type compounds, such as Heliopsis longipes (root), Drymis winteri (bark), Polygonum hydropiper L. (water pepper), Tasmannia lanceolata (Tasmanian Pepper), Tasmannia stipitata (dorrigo pepper), Zanthoxylum rubescens, Zanthoxylum americanum Mill. (prickly ash), Capsicum frutescens, Capsicum chinense, Capsicum pubescens, Zanthoxylum piperitum (Japanese pepper), Spilantha spp. (Paracress), Zingiber officinale Roscoe (ginger root), Syzygium aromaticum L. (clove), Ocimum gratissimum L. (clove basil), Cinnamomum cassia L. (cassia), Cinnamomum zeylanicum Blume (cinnamon), Pimenta dioica [L.] Mer. (allspice), Amomum melegueta (grains of paradise), Piper nigrum L. (black pepper), Kaempferia galanga L. & Alpinia officinarum H. (both of which are referred to as lesser galangal), Alpinia galanga [L.] Wilid. (greater galangal), Piper cubeba L. (cubeb) and/or extractives of such (e.g., capsicum oleoresin, ginger oleoresin, jambu oleoresin, galanga extracts, clove oil, clove oleoresin, cinnamon bark oil, cinnamon bark oleoresin, cubeb oleoresin, cinnamon leaf oil, cassia oil, allspice oil, allspice oleoresin, black pepper oleoresin, etc.). Other natural flavoring materials which possess pungency due to disulfides and trisulfides such as extracts or oils of Allium cepa L. (onion), Allium sativum L. (garlic), Ferula assa-foetida L. (asafoetida), etc. may also be employed.

The present invention is further directed to a method of using such sensate compositions in foods, pharmaceuticals, and personal care products wherein the use of a eutectic cooling mixture of 2-Isopropyl-N,2,3-trimethylbutyramide (WS-23) or N-Ethyl-p-menthane-3-carboxamide (WS-3), or at least one cooling agent selected from the group consisting of N-(2-hydroxyethyl)-2,3-dimethyl-2-isopropylbutanamide. N-(3-ethoxypropyl)-2,3-dimethyl-2-isopropylbutanamide, N-(3-isopropoxypropyl)-2,3-dimethyl-2-isopropylbutanamide, N-(3-butoxypropyl)-2,3-dimethyl-2-isopropylbutanamide,N-Ethyl-2,2-diisopropylbutanamido, N-(1,1-dimethyl-2-hydroxyethyl)-2,2-diethylbutanamide, N-(2-ethoxyethyl)-2,3-dimethyl-2-isopropylbutanamide and N-(3-methoxypropyl)-2,3-dimethyl-2-isopropylbutanamide, in conjunction known pungent, warming or tingling agents provides an enhancement of the pungent, warming or tingling sensation.

For this type evaluation, a panel consisting of both professional flavorists and other individuals with sensory testing experience was employed. In each tasting session two samples were presented to the panelists in a randomized order to avoid presentation order effect. Panelists were asked to rate the samples as same or different. If different, panelists were asked to rate the “pungent, hotness (warming) or tingling” intensity and to describe the flavor profile as compared to a control sample without the eutectic cooling mixture.

### EXAMPLE 1

### Preparation of the Eutectic Liquid Cooling Composition

Fifty grams of 2-Isopropyl-N,2,3-trimethylbutyramide flakes and 50 g of N-Ethyl-p-menthane-3-carboxamide were placed in a 200 ml beaker, and the mixture was heated to above 62° C. to melt the 2-Isopropyl-N,2,3-trimethylbutyramide. The mixture is then stirred until the resulting mixture becomes a clear, transparent liquid composition consisting of 50% of 2-Isopropyl-N,2,3-trimethylbutyramide and 50% N-Ethyl-p-menthane-3-carboxamide. The resulting liquid cooling composition is a eutectic mixture with a crystallization point below 0° and usually −20° centigrade.

### EXAMPLE 2

### Pungency Enhancement of a Hot Chile Pepper Sauce

A commercial hot chile pepper sauce derived from Capsicum frutescens var. tabasco (Tabasco® brand) (whose primary pungent principles are the amides capsaicin and dihydrocapsaicin) was diluted to 5% strength in water (the control). To one portion of this was added 40 ppm of the liquid eutectic cooling composition consisting of 50% of 2-Isopropyl-N,2,3-trimethylbutyramide and 50% N-Ethyl-p-menthane-3-carboxamide prepared as described in Example 1. The control sample was the 5% strength hot chile pepper sauce. Panelists universally agreed that the overall hotness (warmth) and pungency profile was significantly enhanced in the test product while the basic flavor profile remained unchanged. No cooling sensation was described by panelists. The degree of hotness (warmth) and pungency profile enhancement was judged to be 25-35%. In a comparative test, the experiment was repeated, except that only N-Ethyl-p-menthane-3-carboxamide was employed as the cooling agent (at a level of 28 ppm, which cooling strength is comparable to 40 ppm of the described eutectic cooling mixture). In this case, a similar 25-35% increase in overall hotness (warmth) and pungency profile was noted by panelists, but this was accompanied by an unpleasant irritating-scratchy effect, especially in the back of the throat.

Tabasco® Brand is a registered trademark of McIIhenny Company.

### EXAMPLE 3

### Pungency Enhancement of Allyl Isothiocyanate

A water solution containing 10 ppm of allyl isothiocyanate was prepared (the control). To a portion of this was added 40 ppm of the liquid eutectic cooling composition consisting of 50% of 2-Isopropyl-N,2,3-trimethylbutyramide and 50% N-Ethyl-p-menthane-3-carboxamide prepared as described in Example 1.

Panelists universally agreed that the overall hotness (warmth) and pungency profile was significantly enhanced in the test product while the basic flavor profile remained unchanged. No cooling sensation was described by panelists. The degree of hotness (warmth) and pungency profile enhancement was judged to be 90-110%.

### EXAMPLE 4

### Pungency Enhancement of a Hot Mustard Preparation

A commercial Chinese Style Hot Mustard (KA•ME® brand) preparation (whose primary pungent principle is allyl isothiocyanate) was diluted to 25% strength in water (the control). To a portion of this was added 40 ppm of the liquid eutectic cooling composition consisting of 50% of 2-Isopropyl-N,2,3-trimethylbutyramide and 50% N-Ethyl-p-menthane-3-carboxamide prepared as described in Example 1.

Panelists universally agreed that the overall hotness (warmth) and pungency profile was significantly enhanced in the test product while the basic flavor profile remained unchanged. No cooling sensation was described by panelists. The degree of hotness (warmth) and pungency profile enhancement was judged to be 90-110%.

KA•ME® brand is a registered trademark of Tree of Life, Inc.

### EXAMPLE 5

### Pungency Enhancement of a Horseradish Preparation

40 ppm of the liquid eutectic cooling composition consisting of 50% of 2-Isopropyl-N,2,3-trimethylbutyramide and 50% N-Ethyl-p-menthane-3-carboxamide prepared as described in Example 1. was added to a commercial prepared horseradish consisting of horseradish, vinegar and salt (Gold's® brand) and the taste compared to the control horseradish preparation. Panelists universally agreed that the overall hotness (warmth) and pungency profile was significantly enhanced in the test product. In addition, panelists found an increase in the vinegar taste perception. The pungency profile enhancement was judged to be 25-35%.

Gold's® brand is a registered trademark of Gold Pure Food Products Co., Inc

### EXAMPLE 6

### Pungency Enhancement of Horseradish Oil

A water solution containing 7 ppm of horseradish oil (whose primary pungent principles are a series of isothiocyanates of which allyl isothiocyanate and phenylethyl isothiocyanate are the predominant components) was prepared (the control). To a portion of this was added 40 ppm of the liquid eutectic cooling composition consisting of 50% of 2-Isopropyl-N,2,3-trimethylbutyramide and 50% N-Ethyl-p-menthane-3-carboxamide prepared as described in Example 1.

Panelists universally agreed that the overall hotness (warmth) and pungency profile was significantly enhanced in the test product while the basic flavor profile remained unchanged. No cooling sensation was described by panelists. The degree of hotness (warmth) and pungency profile enhancement was judged to be 70-80%.

1. A sensate composition with enhanced pungent, warming or tingling properties that is comprised of a liquid cooling sensate consisting of a eutectic mixture of 2-Isopropyl-N,2,3-trimethylbutyramide and N-Ethyl-p-menthane-3-carboxamide, and at least one warming, tingling, pungent principle component that is an isothiocyanate selected from the group consisting of allyl isothiocyanate, phenylethyl isothiocyanate or a natural product that contains at least one warming, tingling, or pungent principle component that is an isothiocyanate selected from the group consisting of allyl isothiocyanate or phenylethyl isothiocyanate.

2. A sensate composition of claim 1. wherein the liquid cooling sensate is a eutectic mixture of 2-Isopropyl-N,2,3-trimethylbutyramide and N-Ethyl-p-menthane-3-carboxamide with a weight ratio of 2-Isopropyl-N,2,3-trimethylbutyramide to N-Ethyl-p-menthane-3-carboxamide in the range of 60:40 to 30:70 where the corresponding crystallization point is below 15° C.

3. A sensate composition of claim 1. wherein the liquid cooling sensate is a eutectic mixture of 2-Isopropyl-N,2,3-trimethylbutyramide and N-Ethyl-p-menthane-3-carboxamide with a weight ratio of 2-Isopropyl-N,2,3-trimethylbutyramide to N-Ethyl-p-menthane-3-carboxamide in the range of 1:1 where the corresponding crystallization point is below 0° C.

4. A sensate composition of claim 1. wherein the liquid cooling sensate is employed at a rate of 20 ppm to 80 ppm in the finished consumer product.

5. (canceled)

6. (canceled)

7. A sensate composition of claim 1. wherein at least one isothiocyanate having warming, tingling, or pungent properties is allyl isothiocyanate.

8. A sensate composition of claim 1. wherein at least one isothiocyanate having warming, tingling, or pungent properties is phenylethyl isothiocyanate.

9. A sensate composition of claim 1. wherein at least one natural product is employed that contains at least one warming, tingling, pungent principle that is an isothiocyanate selected from the group consisting of allyl isothiocyanate or phenylethyl isothiocyanate.

10. A sensate composition of claim 9. wherein the natural product or products containing such isothiocyanates selected from the group consisting of allyl isothiocyanate or phenylethyl isothiocyanate is selected from the group consisting of Brassica oleracea var. capitata (common cabbage), Brassica rapa var. Pekinensis (Pe-tsai Chinese cabbage), Brassica rapa var. chinensis (Pak-choi, Chinese cabbage), Nasturtium officinale (Watercress), Brassica juncea (Brown or Oriental mustard), Wasabi japonica (Japanese Horseradish), Brassica rapa septiceps (Turnip), Brassica oleracea botrytis (Cauliflower), Cochlearia armoracia or Armoracia rusticana (Horseradish), Brassica oleracea gemmifera (Brussels sprout), Raphanus sativus (Radish), or Raphanus raphanistrum (Wild radish) in the form of the raw or dried botanical, oils distilled from the botanical, extractives of the botanical, or processed foods containing the botanical.

11. A sensate composition of claim 9. wherein at least one of the natural products containing such isothiocyanates selected from the group consisting of allyl isothiocyanate or phenylethyl isothiocyanate is Brassica juncea (Brown or Oriental mustard) in the form of the raw or dried botanical, oils distilled from the botanical, extractives of the botanical, or processed foods containing the botanical.

12. A sensate composition of claim 9. wherein at least one of the natural products containing such isothiocyanates selected from the group consisting of allyl isothiocyanate or phenylethyl isothiocyanate is is Cochlearia armoracia or Armoracia rusticana (Horseradish) in the form of the raw or dried botanical, oils distilled from the botanical, extractives of the botanical, or processed foods containing the botanical.

13. (canceled)

14. (canceled)

15. (canceled)

16. (canceled)

17. (canceled)

18. (canceled)

19. (canceled)

20. (canceled)

21. (canceled)
